(19) United States
(12) Patent Application Publication
(10) Pub. No.: US 2026/0174840 A1
(43) Pub. Date: Jun. 25, 2026

(54) Immunogenic Compositions Containing Non-Natural Amino Acids for the Prevention and Treatment of Pneumococcal Infections

(71) Applicant: Inventprise, Inc., Redmond, WA (US)

(72) Inventor: Subhash V. Kapre, Redmond, WA (US)

(73) Assignee: Inventprise, Inc., Redmond, WA (US)

(21) Appl. No.: 19/417,869

(22) Filed: Dec. 12, 2025

Related U.S. Application Data
(60) Provisional application No. 63/737,644, filed on Dec. 21, 2024.

(51) Int. Cl. A61K 39/09; A61K 39/00; A61K 39/385; C07K 14/315

(52) U.S. Cl. CPC A61K 39/092; A61K 39/385; C07K 14/3156; A61K 2039/572; A61K 2039/6037; C07K 2319/00

(57) ABSTRACT

This invention relates to immunogenic compositions for the prevention and treatment of infection due to Streptococcus pneumoniae containing P4 peptide and/or detoxified pneumolysin protein conjugated to a T cell stimulating epitope as a contiguous peptide sequence. In particular, non-natural amino acid sequences inserted between the structures create points of conjugation that do not interfere with the generation of an effective immune response. This invention is also directed to method for the manufacture of these compositions and administration of these compositions to subjects.

### REFERENCE TO RELATED APPLICATIONS

This application claims priority to U.S. Provisional Application No. 63/737,644 filed Dec. 21, 2024, the entirety of which is incorporated.

### BACKGROUND

### 1. Field of the Invention

This invention is directed to immunogenic compositions for the prevention and treatment of infections attributable to Streptococcus pneumoniae containing P4 peptide and/or detoxified pneumolysin protein conjugated to a T cell stimulating epitope as a contiguous peptide sequence. In particular, non-natural amino acid sequences inserted between the sequences create points of conjugation that do not interfere with the generation of an effective immune response. This invention is also directed to methods for the manufacture of these compositions and method for the administration of such compositions to subjects.

### 2. Description of the Background

Streptococcus pneumoniae, often referred to simply as pneumococci, is a Gram-positive, spherical bacteria. The microorganisms are opportunistic pathogens that are responsible for a large spectrum of infections causing both local and systemic diseases including infectious pneumonia, meningitis, otitis media, bacteremia, and sepsis. This pathogenic microorganism infects, among others, cells of the nasopharynx and oropharynx and is a common cause of morbidity and mortality in the world. Symptoms include fever, cough, chills, shortness of breath, chest pain, neck pain, mental confusion, general disorientation, and sensitivity to light.

Mortality from severe pneumonia and sepsis is high in most countries. Antibiotic treatment alone is ineffective in severe infections and does not reduce the overall death rate, at least in the initial period following infection. In susceptible individuals or persons with depressed immune systems, such as the elderly and the young, the bacterium can spread to other locations throughout the body. The pathogen spreads by direct person-to-person contact such as through respiratory droplets and is an important cause of neonatal infections

Of the S. pneumoniae proteins, pneumococcal surface protein A (PspA) and a 53-kd exotoxin, pneumolysin protein (Ply), are often utilized as the antigenic components in vaccines against infection. Peptide P4, a 28-amino-acid peptide derived from the functional site of the PspA is an immunoactive portion. This peptide (P4) alone enhances phagocytosis with pathogen-specific antibodies in pooled immunoglobulin (IVIG). P4 peptide treatment of macrophages in culture increases the ability of the macrophages to phagocytose S. pneumoniae microorganisms.

Pneumolysin protein is a ubiquitous virulence factor that shows cytolytic activity and stimulates complement, contributes to pore formation of the infected cell, and interferes with host clearance of bacteria by phagocytic and immune cell function. Pneumolysin localizes into the mitochondrial membrane of host cells altering membrane potential leading to the production of apoptosis-inducing factor and cell death receptors.

Pneumolysin contributes to the inflammatory response, plays a role in acute lung injury and the development of biofilms. Ply was also shown to be involved in the mechanism of immunomodulation and, correspondingly, antibodies to Ply can provide a protective role. Ply has a highly conserved amino acid sequence across different strains, but was not considered as a vaccine candidate due to its intrinsic cytolytic activity. Designing a Ply candidate vaccine antigen with the appropriate detoxification (dPly) and immune profile was had limited success. Site-directed mutagenesis produced Ply mutants with reduced hemolytic activity. Toxic activity can also be reduced with formaldehyde treatment.

A number of vaccines are available against pneumococcal diseases. Current vaccines are based on pneumococcal capsular polysaccharides (PS). The capsular polysaccharides of the dominant disease-causing serotypes are used as the antigenic component of the vaccine which is often conjugated with carrier proteins such as CRM (diphtheria toxoid as the cross-reactive material).

The combination of S. pneumoniae PS including those containing PspA, P4 and/or Ply can be protective against infection from a number of different strains of S. pneumoniae. This protection was shown to correspond antibody-mediated C3 deposition. C3 is the most abundant complement protein in serum and antibody-mediated C3 deposition is a process whereby C3 fragments are covalently attached to target tissue after the complement system is fully activated with an increase of opsonophagocytic activity.

Although current vaccines are effective against infection, most tend to lose efficacy overtime due in part to the emergence of new serotypes. Also, conjugate vaccines have high production costs, which greatly curtails their use in developing countries, where the disease is prevalent. New treatments are urgently needed to overcome the morbidity and mortality attributed to infections of S. pneumoniae.

### SUMMARY OF THE INVENTION

The present invention overcomes the problems and disadvantages associated with current strategies and designs and provides new compositions and method directed to immunogenic compositions containing PspA, P4 and/or Ply coupled as a contiguous sequence to T cell epitopes containing non-natural amino acids.

One embodiment of the invention is directed to immunogenic compositions containing as an antigenic portion, which comprise all or a portion of a detoxified pneumolysin peptide, a P4 peptide, and/or a PspA peptide, a T cell stimulating portion, and one or more nnAA (non-naturally occurring amino acids) between the portions. Preferably the dPly peptide and P4 peptide are coupled in a contiguous sequence with a T cell portion, having the nnAA between. Preferably, non-natural amino acid sequences are present between the antigenic and T cell portions so as not to hinder the immune response generated from administration of the composition to a subject, such as the T-helper cell effect, which can increase the resulting immune response. Preferably, polysaccharides can be conjugated to the nnAAs forming a conjugate vaccines to multiple different serotypes.

Compositions of the invention may include nucleic acid sequences that encode the proteins disclosed herein. Preferably, the immunogenic composition of the disclose contain one or more points of conjugation created by the incorporation of non-natural amino acids for the attachment of other antigens. Preferably, the presence of non-natural amino acids allows for efficient antigen presentation to the immune system.

Another embodiment of the invention is directed to methods for the administration of immunogenic compositions of the invention to subjects who are infected or at risk of becoming infected with S. pneumoniae. Compositions may be peptide or nucleic acid sequences, either can be administered as effective vaccines.

Another embodiment of the invention is directed to methods for the manufacture of immunogenic compositions containing detoxified pneumolysin and, optionally P4 peptide sequences, coupled to T cell stimulating epitopes along a contiguous peptide sequence.

Other embodiments and advantages of the invention are set forth in part in the description, which follows, and in part, may be obvious from this description, or may be learned from the practice of the invention.

### DESCRIPTION OF THE INVENTION

Infections attributed to the microorganism Streptococcus pneumoniae are responsible for a great deal of disease and mortality throughout the world. Antibiotic treatment alone is not effective in many situations and does not reduce the overall death rate.

Although a number of vaccines are available against Streptococcus pneumoniae infections, current vaccines are based on pneumococcal capsular polysaccharides (PS). The capsular polysaccharides of the dominant disease-causing serotypes are used as the antigenic component of the vaccine which is often conjugated with carrier proteins. A number of effective vaccines contain PspA, P4 and/or Ply proteins. Although protection can be effective, these vaccines lose efficacy overtime due in part to the emergence of new serotypes. Also, conjugate vaccines have high production costs, which greatly curtails their use in developing countries, where the disease is prevalent. New treatments are urgently needed to overcome the morbidity and mortality attributed to infections of S. pneumoniae.

It has been surprisingly discovered that pneumolysin, a single polypeptide chain composed of 471 amino acids, can be detoxified and utilized as an effective immunogenic composition. Detoxified pneumolysin may be synthesizing by in vitro chemical synthesis, solid-phase protein synthesis, and in vitro (cell-free) protein translation, or recombinantly engineered and expressed in bacterial cells, fungi, insect cells, mammalian cells, virus particles, yeast, and the like. Preferably, pneumolysin is detoxified genetically by removal of the nucleic acid portion that is responsible for creating the toxic effect of the protein and the dPly expressed recombinantly. Preferably the dPly contains a single amino acid substitution of Trp433Phe.

It was further discovered that dPly can be coupled to PspA and/or P4 protein to be utilized as the antigenic portion of immunogenic compositions. Coupling can be ionic or covalent and the antigenic portion is preferable a contiguous peptide sequence. As with many effective immunogenic compositions, coupling the antigenic component with a T cell stimulating portion and/or a TLR agonist, increases the effectiveness in stimulating the immune system. Preferably the T cell stimulating portion is obtained or derived from tetanus toxin, tetanus toxin heavy chain proteins, diphtheria toxoid, CRM, recombinant CRM, tetanus toxoid, Pseudomonas exoprotein A, Pseudomonas aeruginosa toxoid, Bordetella pertussis toxoid, Clostridium perfringens toxoid, Escherichia coli heat-labile toxin B subunit, Neisseria meningitidis outer membrane complex, Hemophilus influenzae protein D, Flagellin Fli C, Horseshoe crab Haemocyanin, and/or an effective fragment, derivative, or modification thereof. These immunogenic compositions may be utilized for treatment and/or prevention of infection as vaccines.

It was further discovered that, by inserting one or more non-naturally occurring amino acids (nnAA) between the sequences of the antigenic portion and the T cell stimulating portion, there is minimal to no hinderance of the immunogenic activity of either portion. nnAAs can be inserted through recombinant technology, or the sequence may be chemically synthesized. Preferably, one or more nnAAs are incorporated recombinantly into the peptide sequence through nonsense and/or missense nonsense codons (e.g., amber, ochre, or opal codons) that were specifically introduced. Preferably, the non-naturally occurring amino acids are incorporated between the antigenic and T cell sequences. Preferably, number of non-naturally occurring amino acids incorporated into a particular protein is 10 or less, 9 or less, 8 or less, 7 or less, 6 or less, 5 or less, 4 or less, 3 or less, 2 or less, or only one. Preferably the site along the peptide sequence at which the non-naturally occurring amino acids are incorporated is not an enzymatically active site, or a structurally important site of the immunogenic composition. Preferably the site of incorporation is convenient for conjugation. Again, these immunogenic compositions may be utilized for treatment and/or prevention of infection as vaccines.

The presence of nnAA provide convenient sites for the attachment of additional antigenic molecules, such as, for example, capsular polysaccharides of microorganisms. These microorganisms may be one or more species or serotype of Streptococcus pneumoniae. Preferably attachment is via conjugation chemistry and multiple PS or multiple different serotypes can be included in a single composition. Preferably the conjugated composition retain both the antigenic and T cell stimulating activities as the unconjugated compositions. PSs of other non-Streptococcus microorganisms may be added as well such as other species and/or serotypes of Streptococcus. Preferably other microbes include, but is not limited to, PSs of one or more species and/or serotype of Corynebacterium, Clostridium, Haemophilus influenzae, Pseudomonas, and/or Staphylococcus, for example. Immunogenic compositions of this disclosure may include, but are not limited to 5 or more different PSs, 10 or more different PSs, 15 or more different PSs, 20 or more different PSs, 25 or more different PSs, 30 or more different PSs, 35 or more different PSs, 40 or more different PSs, 45 or more different PSs, 50 or more different PSs, 55 or more different PSs, 60 or more different PSs, 65 or more different PSs, or 70 or more different PSs from different species and/or serotypes, which may be of the same or different microbe. Such compositions are preferably multivalent compositions and vaccines.

There are about 20 naturally occurring amino acids, which include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. Examples of nnAA include, but are not limited to hydroxyproline (Hyp), beta-alanine, citrulline (Cit), ornithine (Orn), norleucine (Nle), 3-nitrotyrosine, nitroarginine, pyroglutamic acid (Pyr), moieties bearing an azide, tetrazine, trans-cyclooctene (TCO), aza-dibenzocyclooctyne (DBCO), bicyclononyne (BCN), aminooxy, hydrazides, hydrazines, arylpropiolonitrile (APN) or propargyle groups, and also chemically synthesized amino acids. The incorporation of nnAA into the recombinant molecule creates sites along the peptide chain which can facilitate the application of click chemistry, the rapid and efficient joining of moieties of interest. The placement of the nnAA into the recombinant molecule of interest can be at most any location along the amino acid sequence. Nonsense codons cause premature termination of translation and missense nonsense codons cause the introduction of an incorrect (or different) amino acid into the translated polypeptide sequence. For example, nnAAs are incorporated into a recombinant molecule co-translationally using an engineered, orthogonal transfer RNA (tRNA) that recognizes the amber stop codon (UAG), ochre stop codon (UAA), opal stop codon (UGA), or other nonsense codons (codons for which no normal tRNA molecule exists), and/or missense nonsense codons, as well as an aminoacyl-tRNA synthetase (RS) that catalyzes the aminoacylation of the orthogonal tRNA with the nnAA. Orthogonal aminoacyl tRNA synthetase (aaRS)/tRNA pairs facilitate site-specific introduction of para-azido-L-phenylalanine (pAzF), a non-canonical amino acid, into the amber codon (the UAG codon which causes termination of protein translation) of proteins. Particularly useful nnAA are ones containing an azide group, which facilitate “click reactions” with an alkyne. Click reactions involve the chemical synthesis of biocompatible chemical moieties that form quickly, and in high yields with minimal reaction byproducts. In a preferred click reaction, a partner moiety, for example a PEG molecule or a saccharide or polysaccharide, derivatized with a constrained alkyne, easily reacts with the azide. Thus, the incorporation of site-specific azide amino acids allows for site-specific conjugation.

Another embodiment of the invention is directed to polynucleotides including DNA, RNA (e.g., cRNA, mRNA), and PNA (peptide nucleic acid) constructs that encode the composite sequences of the invention. These polynucleotides may be single-stranded (coding or antisense) or double-stranded, and may be DNA (genomic, cDNA or synthetic) or RNA molecules. Additional coding or non-coding sequences may, but need not, be present within a polynucleotide of the present invention, and a polynucleotide may, but need not, be linked to other molecules and/or support materials. As is appreciated by those of ordinary skill in the art that, as a result of the degeneracy of the genetic code, there are many nucleotide sequences that encode a given primary amino acid sequence. Some of these polynucleotides bear minimal homology to the nucleotide sequence of any native gene. Nonetheless, polynucleotides that vary due to differences in codon usage are specifically contemplated by the present invention. Polynucleotides that encode an immunogenic peptide may generally be used for production of the peptide, in vitro or in vivo. Any polynucleotide may be further modified to increase stability in vivo. Possible modifications include, but are not limited to, the addition of flanking sequences at the 5′ and/or 3′-ends; the use of phosphorothioate or 2′-o-methyl rather than phosphodiesterase linkages in the backbone; and/or the inclusion of nontraditional bases such as inosine, queosine and wybutosine, as well as acetyl-methyl-, thio- and other modified forms of adenine, cytidine, guanine, thymine and uridine.

A nucleic acid vaccine that translates to the compositions of the invention contains the genetic sequence of a composite antigen as cRNA or mRNA, or DNA, plus other necessary sequences that provide for the expression of the composite antigen in cells. By injecting the mammal with the genetically engineered nucleic acid, the composite antigen is produced in or preferably on cells, which the mammal's immune system recognizes and thereby generates a humoral or cellular response to the composite antigen, and therefore the pathogen. Nucleic acid vaccines have a number of advantages over conventional vaccines, including the ability to induce a more general and complete immune response in the mammal. Accordingly, nucleic acid vaccines can be used to protect an animal or a mammal against disease caused from many different pathogenic organisms of viral, bacterial, and parasitic origin as well as certain tumors.

Nucleic acid vaccines encode an antigen contained in vectors or plasmids that have been genetically modified to transcribe and translate the peptide sequences in accordance with the compositions disclosed herein. By way of example, the nucleic acid vaccine is administered, and the cellular machinery transcribed and/or translates the nucleic acid into the antigens which produce an immune response. The antigens, being non-natural and unrecognized by the mammalian immune system, are processed by cells and the processed proteins, preferably the epitopes, displayed on cell surfaces. Upon recognition of these antigens as foreign, the immune system generates an appropriate immune response that protects from the infection. In addition, nucleic acid vaccines of the invention are preferably codon optimized for expression in the animal (or mammal) of interest. In a preferred embodiment, codon optimization involves selecting a desired codon usage bias (the frequency of occurrence of synonymous codons in coding DNA) for the particular cell type so that the desired peptide sequence is expressed.

Immunogenic compositions of the invention may contain adjuvants such as, for example, a toll-like receptor agonist (TLR), Freund's adjuvant, ALFQ, ALFQA, ALFA, AS01, AS01b, a liposome adjuvant, saponin, lipid A, squalene, emulsions, nano-emulsions, or effective derivatives or effective combinations thereof. The formulation of pharmaceutically-acceptable excipients and carrier solutions is well known to those of ordinary skill in the art, as is the development of suitable dosing and treatment regimens for using the particular compositions described herein in a variety of treatment regimens.

The amount of immunogenic composition(s) and the time needed for the administration of such immunogenic composition(s) will be within the purview of the ordinary-skilled artisan having benefit of the present teachings. The administration of a therapeutically-effective, pharmaceutically-effective, and/or prophylactically-effective amount of the disclosed immunogenic compositions may be achieved by a single administration. Alternatively, in some circumstances, it may be desirable to provide multiple, or successive administrations of the immunogenic compositions, either over a relatively short, or even a relatively prolonged period of time, as may be determined by the skilled person overseeing the administration of such compositions.

The immunogenic compositions and vaccines of the present invention may be administered IM, SQ, Intradermal, or intranasal or in a manner compatible with the dosage formulation, and in such an amount as will be prophylactically or therapeutically effective and preferably immunogenic. The quantity to be administered depends on the subject to be treated, including, e.g., the capacity of the immune system to mount an immune response, and the degree of protection desired. Suitable dosage ranges may be on the order of several hundred micrograms (μg) of active ingredient per animal or mammal with a preferred range from about 0.1 μg to 2000 μg (even though higher amounts, such as, e.g., in the range of about 1 to about 10 mg are also contemplated), such as in the range from about 0.5 μg to 1000 μg, preferably in the range from about 1 μg to about 500 μg and especially in the range from about 10 μg to about 100 μg. Suitable regimens for initial administration and booster shots are also variable but are typified by an initial administration followed by optional but preferred subsequent inoculations or other periodic administrations.

An effective dose comprises amounts the range of about 0.1 μg to about 1 mg total protein or target antigen per animal or mammal. In one exemplary embodiment, the vaccine dosage range is about 0.1 μg to about 10 mg per animal or mammal. However, one may prefer to adjust dosage based on the amount of peptide delivered. In either case, these ranges are merely guidelines from which one of ordinary skill in the art may deviate according to conventional dosing techniques. Precise dosages may be determined by assessing the immunogenicity of the immunogenic composition produced in the appropriate host so that an immunologically effective dose is delivered. An immunologically effective dose is one that stimulates the immune system of the animal or mammal to establish an immune response to the immunogenic composition or vaccine. Preferably, the level of immunological memory is sufficient to provide long-term protection against infection. The immunogenic compositions or vaccines of the invention may be preferably formulated with an adjuvant. By “long-term” it is preferably meant over a period of time of at least about 6 months, over at least about 1 year, over at least about 2 to 5 or even at least about 2 to about 10 years or longer. Preferably protection is provided with one administration (or one initial series of administrations) and multiple administrations over time are not required.

Other embodiments and uses of the invention will be apparent to those skilled in the art from consideration of the specification and practice of the invention disclosed herein. All references cited herein, including all publications, U.S. and foreign patents and patent applications, are specifically and entirely incorporated by reference. It is intended that the specification and examples be considered exemplary only with the true scope and spirit of the invention indicated by the following claims. Furthermore, the term “comprising of” includes the terms “consisting of” and “consisting essentially of.”

1. An immunogenic composition containing a contiguous amino acid sequence comprising:
an antigenic portion comprised of part or all of a PspA (pneumococcal surface protein A) peptide, a P4 (functional portion PspA) peptide, and/or a dPly (detoxified pneumolysin) peptide:
a T cell stimulating portion; and
one or more non-naturally occurring amino acids (nnAA) between the antigenic portion and the T cell stimulating portion.

2. The immunogenic composition of claim 1, wherein the antigenic portion contains P4 peptide coupled to dPly peptide.

3. The immunogenic composition of claim 1, wherein the antigenic portion contains PspA peptide coupled to dPly peptide.

4. The immunogenic composition of claim 1, wherein the antigenic portion contains PspA peptide coupled to P4 peptide.

5. The immunogenic composition of claim 1, wherein the T cell stimulation portion contains a peptide selected from the group consisting of tetanus toxin, tetanus toxin heavy chain proteins, diphtheria toxoid, CRM, recombinant CRM, tetanus toxoid, and an effective fragment, derivative, or modification thereof.

6. The immunogenic composition of claim 1, wherein the one or more non-naturally occurring amino acids are selected from the group consisting of hydroxyproline (Hyp), beta-alanine, citrulline (Cit), ornithine (Orn), norleucine (Nle), 3-nitrotyrosine, nitroarginine, pyroglutamic acid (Pyr), moieties bearing an azide, tetrazine, trans-cyclooctene (TCO), aza-dibenzocyclooctyne (DBCO), bicyclononyne (BCN), aminooxy, hydrazides, hydrazines, arylpropiolonitrile (APN) or propargyle groups, and chemically synthesized amino acids.

7. The immunogenic composition of claim 1, which contains one non-naturally occurring amino acid.

8. The immunogenic composition of claim 1, which contains two non-naturally occurring amino acids.

9. The immunogenic composition of claim 1, which contains more than two non-naturally occurring amino acids.

10. The immunogenic composition of claim 1, wherein immunogenic activity the antigenic portion does not interfere with the immunogenic activity of the T cell portion.

11. The immunogenic composition of claim 1, which further contains one or more polysaccharides conjugated to one or more of the nnAA.

12. The immunogenic composition of claim 11, wherein the one or more polysaccharides comprise one or more different serotype and/or species of Streptococcus.

13. The immunogenic composition of claim 11, wherein the one or more polysaccharides comprise one or more serotypes and/or species of microorganisms other than Streptococcus.

14. A method for the manufacture of the immunogenic composition of claim 1, comprised of:
recombinantly constructing a contiguous peptide sequence containing the antigen portion coupled to the T cell stimulating portion that contains nnAA between the portions.

15. A method for treating or preventing a species of Streptococcal infection by administering the immunogenic composition of claim 1 to a subject.

16. The method of claim 15, wherein the spp. of Streptococcal infection is Streptococcus pneumoniae.

17. An immunogenic composition containing a contiguous amino acid sequence comprising:
an antigenic portion containing a dPly peptide coupled to a P4 peptide; and
one or more non-naturally occurring amino acids.

18. The composition of claim 17, further comprising a T cell stimulating portion.

19. The composition of claim 18, wherein the one or more non-naturally occurring amino acids are between the antigenic portion and the T cell stimulating portion.
